(19) United States
(12) Patent Application Publication
(10) Pub. No.: US 2008/0051634 A1
(43) Pub. Date: Feb. 28, 2008

(54) Endoscope

(76) Inventors: Tomoaki Yamashita, Hachioji-shi (JP); Hiroyuki Nagamizu, Sagamihara-shi (JP); Yasuyuki Futatsugi, Hachioji-shi (JP)

Correspondence Address: OSTROLENK FABER GERB & SOFFEN, 1180 AVENUE OF THE AMERICAS, NEW YORK, NY, 100368403, US

(21) Appl. No.: 11/571,193

(22) PCT Filed: Jun. 24, 2005
(86) PCT No.: PCT/JP05/11633

§ 371 Date: Dec. 22, 2006

(30) Foreign Application Priority Data
Jun. 24, 2004 (JP) 2004-086349

(51) Int. Cl. A61B 1/018; A61B 1/00

(52) U.S. Cl. 600/134; 600/153

(57) ABSTRACT

An outer sheath of a metal film is provided on an outer peripheral surface of a tube base layer of a channel tube which is provided within an insertion section and has flexibility. A grounding circuit provided in an operation section and the metal film are connected, and electromagnetic shield means is provided for reducing noise which is produced from a radio-frequency therapeutic device inserted in a therapeutic device insertion channel and is mixed in an electric signal output from an imaging section.

### CROSS-REFERENCE TO RELATED APPLICATIONS

This is a Continuation Application of PCT Application No. PCT/JP2005/011633, filed Jun. 24, 2005, which was published under PCT Article 21(2) in Japanese.

This application is based upon and claims the benefit of priority from prior Japanese Patent Application No. 2004-186349, filed Jun. 24, 2004, the entire contents of which are incorporated herein by reference.

### BACKGROUND OF THE INVENTION

1. Field of the Invention

The present invention relates to an endoscope including an imaging element, such as a CCD, for capturing an endoscopic image, and a therapeutic device insertion channel, the endoscope performing a radio-frequency therapeutic treatment by means of a radio-frequency therapeutic device that is inserted in the therapeutic device insertion channel.

2. Description of the Related Art

In general, in an electronic endoscope (videoscope), an imaging element, such as a CCD, is incorporated in a distal end portion of an insertion section that is inserted in a lumen, and an endoscopic image is captured by the imaging element. The endoscope is provided with a signal cable which transmits a signal that is output from the imaging element. The signal cable is connected to an external camera control unit (CCU). The CCU is connected to display means such as a monitor. An endoscopic image that is captured by the imaging element is converted to an electric signal and transmitted to the CCU via the signal cable, and thus the endoscope image is displayed on the monitor.

A forceps channel (therapeutic device insertion channel) is provided in the insertion section of the endoscope. A radio-frequency therapeutic device, for instance, is inserted in the forceps channel. The radio-frequency therapeutic device is inserted into the body via the forceps channel, and radio-frequency therapeutic treatment is performed in the body.

Japanese Patent No. 2997797 (Patent Document 1) discloses a structure wherein a mesh tube, which is formed by weaving metal wires into a mesh, is laid over an outer sheath of an insertion section of an endoscope or an outer sheath of a universal cable. With this structure, undesired radiation noise from the electronic endoscope is reduced, or noise radiated from an external electronic device is prevented from entering the electronic endoscope.

Jpn. Pat. Appln. KOKOKU Publication No. 7-61308 (Patent Document 2) discloses a structure wherein a metal foil of, e.g. aluminum is wrapped over the outer periphery of a channel tube of a forceps channel, which is provided in an insertion section of an endoscope, and a metal evaporation-deposition coating film, which is formed by evaporation deposition of a metal such as copper, is provided on the metal foil.

Jpn. Pat. Appln. KOKAI Publication No. 8-256974 (Patent Document 3) discloses a structure wherein dual-layer shield wires are wound around an image-capturing cable, which is a signal cable connected to an operation section of the endoscope, thereby preventing electromagnetic interference on an external electric device. In this case, too, undesired radiation noise from the electronic endoscope is reduced, or noise radiated from an external electronic device is prevented from entering the electronic endoscope.

### BRIEF SUMMARY OF THE INVENTION

According to a first aspect of the present invention, there is provided an endoscope comprising an insertion section which includes a distal end portion and a proximal end portion and is inserted in a lumen, a therapeutic device insertion channel which is provided in the insertion section and permits passage of a therapeutic device, an imaging section which is provided at the distal end portion of the insertion section, captures an image of a subject, converts the captured image of the subject to an electric signal, and outputs the electric signal and electromagnetic shield means which is provided in the therapeutic device insertion channel and suppresses, when noise is produced from the therapeutic device inserted in the therapeutic device insertion channel, the noise from mixing in the electric signal that is output from the imaging section.

According to a second aspect of the present invention, there is provided an endoscope comprising an insertion section which includes a distal end portion and a proximal end portion and is inserted in a lumen, a channel tube constituting a therapeutic device insertion channel which is provided in the insertion section and permits passage of a therapeutic device, an imaging section which is provided at the distal end portion of the insertion section, captures an image of a subject, converts the captured image of the subject to an electric signal, and outputs the electric signal and a metallic part which is provided on the channel tube, wherein the metallic part is connected to a ground on a circuit.

The insertion section may be provided with a bend portion which is bent in accordance with a user's operation, and the metallic part includes a metallic coil member provided on a part of the channel tube, which corresponds to the bend portion, and a metal coating film provided on a part of the channel tube, which is other than the part corresponding to the bend portion, the coil member and the metal coating film being electrically connected.

The imaging section may be includes an observation optical system and an imaging element, and an image-capturing cable which is connected to the imaging element is inserted in the insertion section.

The channel tube may be includes a tube base layer which is formed of a resin material, and the metal coating film is formed of a plated coating film.

The channel tube may be includes a tube base layer which is formed of a resin material, and the metal coating film includes a film layer in which films of a plurality of kinds of metals are successively formed, and an outermost film layer which is formed of at least one of gold and nickel as an outermost sheath of the film layer.

### BRIEF DESCRIPTION OF THE SEVERAL VIEWS OF THE DRAWING

FIG. 1 schematically shows the structure of the entire system of an endoscope according to a first embodiment of the present invention;

FIG. 2A is a vertical cross-sectional view showing an internal structure of an insertion section of the endoscope according to the first embodiment;

FIG. 2B is a vertical cross-sectional view of a main part, which shows the structure of a metal layer of the outer sheath of the channel tube of the endoscope according to the first embodiment;

FIG. 3 is a vertical cross-sectional view which schematically shows the internal structure of a distal end portion of the insertion section of the endoscope according to the first embodiment;

FIG. 4 is a schematic view of the structure of a main part, which shows the connection state of a grounding circuit of an endoscope according to a second embodiment of the invention;

FIG. 5 is a vertical cross-sectional view showing the internal structure of an insertion section of an endoscope according to a third embodiment of the invention;

FIG. 6 is a vertical cross-sectional view of a main part, which shows a connection part between a coil member and a metal film of the endoscope according to the third embodiment;

FIG. 7 is a vertical cross-sectional view of a main part, which shows a first modification of the connection part between the coil member and the metal film of the endoscope according to the third embodiment;

FIG. 8 is a vertical cross-sectional view of a main part, which shows a second modification of the connection part between the coil member and the metal film of the endoscope according to the third embodiment;

FIG. 9 is a vertical cross-sectional view showing the internal structure of an insertion section of an endoscope according to a fourth embodiment of the invention;

FIG. 10 is a vertical cross-sectional view showing the internal structure of an insertion section of an endoscope according to a fifth embodiment of the invention;

FIG. 11A is a vertical cross-sectional view showing the internal structure of an insertion section of an endoscope according to a sixth embodiment of the invention; and

FIG. 11B is a perspective view which shows a grounding connection part of the endoscope according to the sixth embodiment.

### DETAILED DESCRIPTION OF THE INVENTION

### First Embodiment

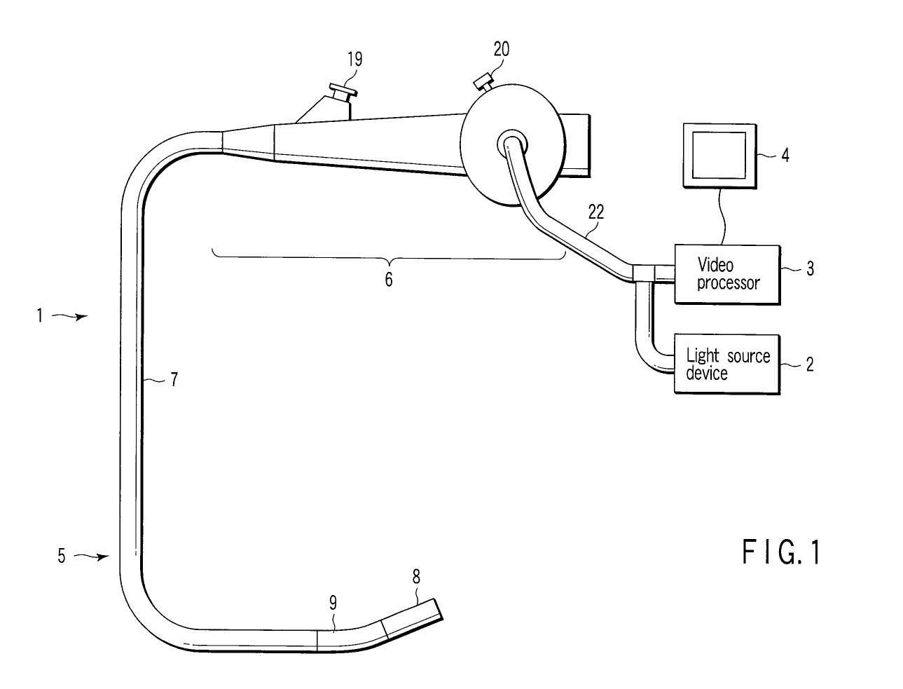

A first embodiment of the present invention will now be described with reference to FIG. 1 to FIG. 3. FIG. 1 schematically shows the structure of the entire system of an electronic endoscope (videoscope) 1 according to the present embodiment. The system of the electronic endoscope 1 includes the electronic endoscope 1, a light source device 2, a video processor 3, and a monitor 4. The monitor 4 is connected to the video processor 3.

The electronic endoscope 1 includes an elongated insertion section 5 which is inserted in a lumen, and an operation section 6 which is connected to a proximal end portion of the insertion section 5. The insertion section 5 is provided with an elongated flexible tube portion (flexible insertion tube) 7 having flexibility. A proximal end portion of the flexible tube portion 7 is connected to the operation section 6. A distal end portion of the insertion section 5 is provided with a distal-end rigid portion 8. A bend portion 9 is interposed between a proximal end portion of the distal-end rigid portion 8 and a distal end portion of the flexible tube portion 7.

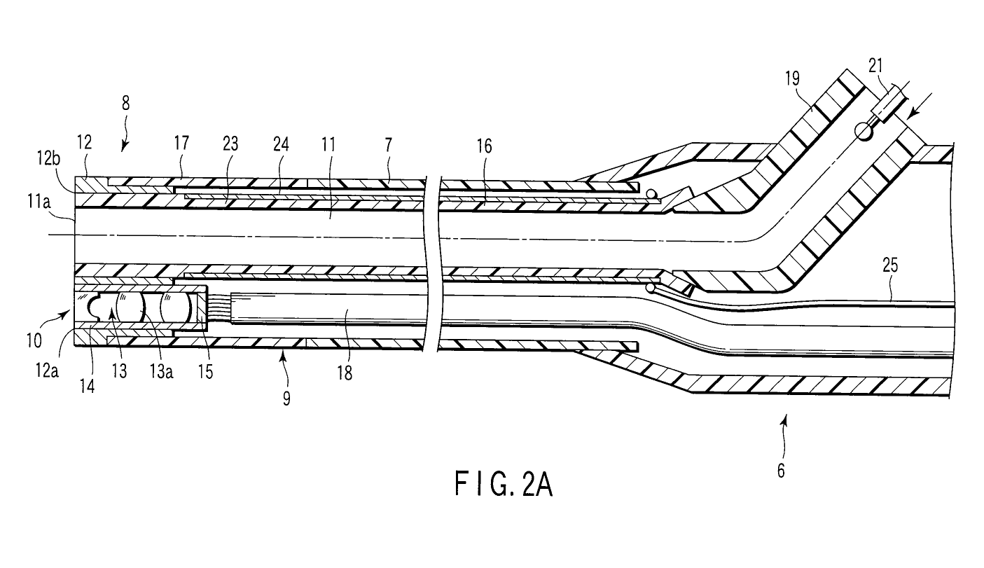

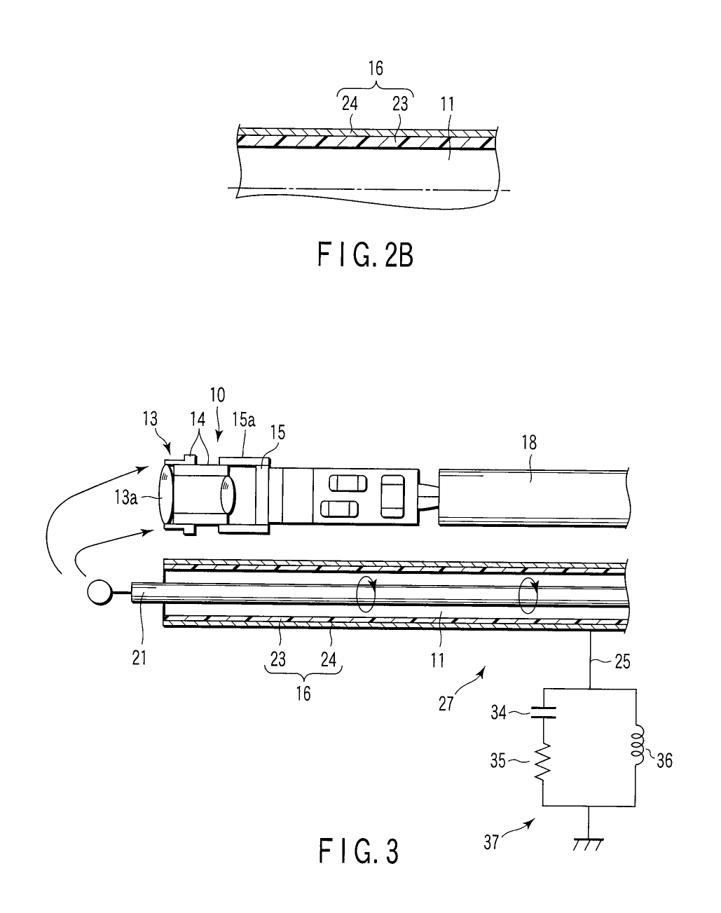

As is shown in FIG. 2A, the distal-end rigid portion 8 is provided with at least an observation section 10, a distal-end opening 11a of a therapeutic device insertion channel 11, and an illumination section (not shown). Further, the distal-end rigid portion 8 is provided with a distal-end frame 12. The distal-end frame 12 is provided with an observation section attachment hole 12a, a hole 12b for the therapeutic device insertion channel, and a hole (not shown) for illumination.

The observation section 10 is provided with an objective lens unit 13 in which objective lenses 13a are incorporated. A CCD hold frame 14, which holds a field lens 200 and a CCD (imaging element) 15, is connected to the objective lens unit 13. The CCD 15 is provided at a rear end part of the CCD hold frame 14. This CCD 15 is disposed at a focal position of the objective lenses 13a. The CCD hold frame 14 is inserted in the observation section attachment hole 12a of the distal-end frame 12 and is, in this state, liquid-tightly fixed by an adhesive or the like.

An illumination unit, which incorporates an illumination lens, a light guide fiber, etc. of an illumination optical system, is mounted in the hole for illumination of the distal-end frame 12. The illumination unit is similarly liquid-tightly fixed in the distal-end frame 12 by an adhesive or the like.

The therapeutic device insertion channel 11 is formed by an elongated channel tube (tubular member) 16 having flexibility. The channel tube 16 is formed of a resin material such as PTFE. A distal end portion of the channel tube 16 is inserted in the therapeutic device insertion channel hole 12b of the distal-end frame 12 and is, in this state, liquid-tightly fixed by an adhesive or the like.

In the bend portion 9 of the insertion section 5, a plurality of bend pieces (not shown) are arranged in the axial direction of the insertion section 5. Front and rear end portions of the respective bend pieces are rotatably coupled. Further, a flexible bend tube 17 is provided on the outer periphery of the bend portion 9. The bend portion 9 can be bent by a remote-control operation from the proximal-end side, for example, in four directions, that is, front, back, right and left directions, or two of these directions.

An image-capturing cable 18, a channel tube 16, a light guide fiber (not shown) for illumination, and an operation wire for a bending operation are provided in the bend portion 9 and flexible tube portion 7 of the insertion section 5. A distal end portion of the image-capturing cable 18 is connected to the CCD 15. In addition, a distal end portion of the operation wire is connected to the foremost bend piece of the bend portion 9. Further, proximal end portions of the image-capturing cable 18, channel tube 16, light guide fiber and operation wire extend to the operation section 6 side.

A channel mouthpiece 19, which is a forceps opening, and a bend operation lever 20 are provided in the operation section 6. As is shown in FIG. 2A, a proximal end portion of the channel tube 16 is coupled to an inner end portion of the channel mouthpiece 19. Thereby, a therapeutic device, such as radio-frequency therapeutic device 21, which is inserted from the channel mouthpiece 19, is guided to the distal end side of the insertion section 5 through the therapeutic device insertion channel 11 and is let to project from the distal-end opening 11a to the outside.

The bend operation lever 20 is coupled to a bend operation mechanism (not shown) which is incorporated in the operation section 6. A proximal end portion of the operation wire is coupled to the bend operation mechanism. The operation wire is pulled via the bend operation mechanism by the operation of the bend operation lever 20, and the bend portion 9 is bent by a remote control in the direction of operation of the bend operation lever 20.

Further, a proximal end portion of a universal cable 22 is connected to the operation section 6. A distal end portion of the universal cable 22 is provided with a connector (not shown) which is detachably connected to the light source device 2, and an electric connector (not shown) which is detachably connected to the video processor 3. Illumination light, which is emitted from the light source device 2, is sent to the illumination section (not shown) of the distal-end rigid portion 8 via the light guide fiber (not shown), and the illumination light is emitted from the illumination section to the outside.

A proximal end portion of the image-capturing cable 18 extends from the operation section 6 through the universal cable 22, and is connected to the electric connector (not shown). An endoscopic image (an image of a subject), which is focused by the objective lenses 13a of the observation section 10, is captured by the CCD 15, and the captured image of the subject is converted to an electric signal. Further, the electric signal that is output from the CCD 15 is sent to the video processor 3 via the image-capturing cable 18, and subjected to image processing. Thus, the endoscopic image is displayed on the monitor 4.

As is shown in FIG. 2B, the channel tube 16 in this embodiment is configured such that an outer sheath 24, which is composed of a single-layer electrically conductive metal film, is provided on the outer periphery of a tube base layer 23 that is composed of a resin material such as PTFE. The tube base layer 23 may be formed of a high-polymer, resin material, other than PTFE, such as FEP, vinyl chloride, PET, polyamide or polyimide. The metal film is provided within the flexible tube portion 7 over a region where the tube base layer 23 extends along the image-capturing cable 18 (i.e. within the flexible tube portion 7 on the proximal-end side of the flexible tube portion 7 from the position of the CCD).

The condition for forming the metal film of the outer sheath 24 is that a metal film of, e.g. copper, chromium, nickel or titanium is formed by CVD, sputtering, evaporation deposition, plating, etc. The method of plating of the metal film of the outer sheath 24 is, for instance, evaporation deposition, sputtering, ion plating or CVD as dry plating. In addition, coating painting (electrically conductive paint material) is applicable as wet plating.

In a pre-treatment for the formation of the metal film of the outer sheath 24, the outer peripheral surface of the tube base layer 23 is subjected to at least one of tetra-etching, solid sodium process, atmospheric plasma, impartment of hydrophilic properties, spreading of primer, etc. Thereby, the affinity between the tube base layer 23 and the metal is enhanced.

One end portion of a lead line 25 is connected to the metal film of the outer sheath 24 of the channel tube 16. As shown in FIG. 3, the other end portion of the lead line 25 is connected to a ground (GND) within the video processor 3 via a grounding circuit 37 which is composed of a capacitor 34, a resistor 35 and a coil 36 within the video processor 3, thereby forming electromagnetic shield means 27. By electrically connecting the lead line 25 to the ground within the video processor 3, noise occurring from the radio-frequency therapeutic device 21, which is inserted in the channel tube 16, is suppressed from mixing in the output signal from the CCD 15.

In the present embodiment, the CCD hold frame 14 of the objective lens unit 13 of the observation section 10 is formed by using an aluminum member that is subjected to non-conductive alumite treatment. A frame member 15a for holding the CCD 15 is also formed by using an aluminum member that is subjected to non-conductive alumite treatment.

Next, the operation of the above-described structure is described. When the electronic endoscope 1 of this embodiment is used, the therapeutic device, such as radio-frequency therapeutic device 21, is inserted from the channel mouthpiece 19. The radio-frequency therapeutic device 21 is guided to the distal-end side of the insertion section 5 through the therapeutic device insertion channel 11, and is let to project from the distal-end opening 11a to the outside. In this state, the electronic endoscope 1 and radio-frequency therapeutic device 21 are used in combination. At this time, an endoscopic image within a lumen is captured by the CCD 15 in the electronic endoscope 1, and the endoscopic image is displayed on the monitor 4.

In addition, when the radio-frequency therapeutic device 21 is used, an electromagnetic field occurs around the radio-frequency therapeutic device 21. At this time, in this embodiment, since the metal film of the outer sheath 24 of the channel tube 16 is connected to the grounding circuit 26 via the lead line 25, the electromagnetic field occurring around the radio-frequency therapeutic device 21 is suppressed from mixing in the output signal from the CCD 15 by the grounding of the grounding circuit 26. Thus, it is possible to reduce crosstalk with the image-capturing cable 18 connected to the CCD 15 due to the electromagnetic field occurring around the radio-frequency therapeutic device 21. Therefore, disturbance of the endoscopic image displayed on the monitor 4 can further be reduced.

The following advantageous effects can be obtained by the above-described structure. The channel tube 16 of the electronic endoscope 1 of this embodiment is configured such that the outer sheath 24 is formed by providing the metal film on the outer periphery of the tube base layer 23, and thus the device of the embodiment is robust to noise from the radio-frequency therapeutic device 21 that is inserted in the channel tube 16. It is possible, therefore, to suppress disturbance of the observation image of the endoscope 1 due to mixture of noise from the radio-frequency therapeutic device 21 that is inserted in the therapeutic device insertion channel 11 of the insertion section 5.

Furthermore, since the outer sheath 24 of the channel tube 16 is formed of the metal film provided on the outer periphery of the tube base layer 23, the thickness of the entire channel tube 16 does not greatly increase. Advantageously, the outside diameter of the insertion section 5 does not increase, and the flexibility of the insertion section 5 does not deteriorate.

In this embodiment, the CCD hold frame 14 of the objective lens unit 13 of the observation section 10 and the frame member 15a that holds the CCD 15 are formed by using aluminum members that are subjected to non-conductive alumite treatment. For example, even in the case of the electronic endoscope 1 that is used in perfusate or physiological saline, radio-frequency leak current flowing through the perfusate is prevented from flowing to the CCD 15 via the CCD hold frame 14 of the objective lens unit 13. As a result, even in the case where the device of this embodiment is used in combination with the radio-frequency therapeutic device 21 that is inserted in the therapeutic device insertion channel 11 of the insertion section 5, it becomes possible to prevent disturbance of the observation image of the endoscope 1 due to radio-frequency waves propagating through the perfusate or physiological saline.

Further, the CCD hold frame 14 and the frame member 15a of the CCD 15, which are formed of aluminum members that are subjected to non-conductive alumite treatment, are employed within the objective lens unit 13. In the case where a non-conductive member of ceramics is provided within the objective lens unit 13, if the diameter of the electronic endoscope 1 is reduced, the thickness of the ceramics decreases and cracks occur in the processing or assembly of the insulating member. This problem can be prevented in the present embodiment.

The channel tube 16 of the present embodiment is configured such that the outer sheath 24 is formed by providing the single-layer metal film on the outer periphery of the tube base layer 23 that is formed of resin material. Alternatively, electrically conductive mesh wires of copper or stainless steel may be formed on the outer periphery of the channel tube 16, and the mesh wires may be electrically connected to a ground within the endoscope 1.

In this embodiment, the outer sheath 24 is formed by providing the metal film over the entire outer periphery of the tube base layer 23 of the channel tube 16. Alternatively, when the metal film of the outer sheath 24 is formed, a spiral masking member, for instance, may be used and a spiral outer sheath 24 may be formed. Needless to say, the shape of the metal film of the outer sheath 24 is not limited to the above-described embodiment, and the metal film may variously be modified and formed in a mesh shape, a lattice shape, etc. In this case, the flexibility of the channel tube 16 can further be secured.

### Second Embodiment

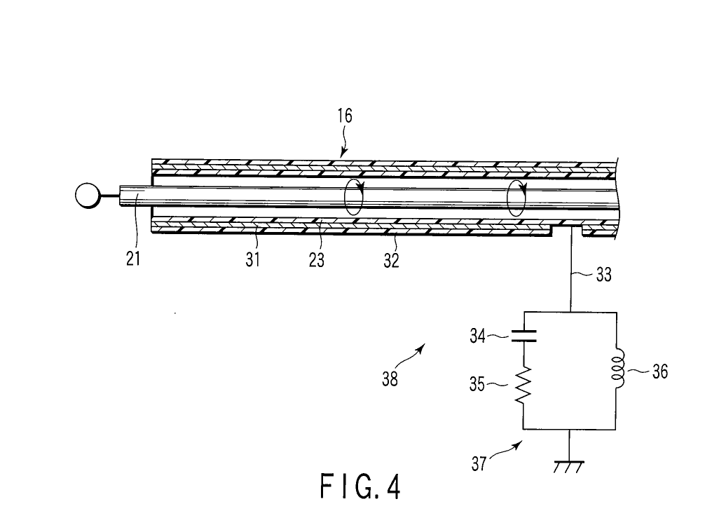

FIG. 4 shows a second embodiment of the present invention. In this embodiment, the structure of the channel tube 16 of the first embodiment (see FIG. 1 to FIG. 3) is altered as described below.

In this embodiment, an intermediate layer 31, which is formed of electrically conductive mesh wires of, e.g. copper or stainless steel, or an electrically conductive metal film of, e.g. aluminum, nickel, copper or gold, is formed on the outer periphery of the tube base layer 23 of the channel tube 16, as in the first embodiment, and a resin layer 32 is formed on the intermediate layer 31. The intermediate layer 31 in this embodiment may be a single-layer metal film, as in the first embodiment, or may be electrically conductive mesh wires of, e.g. copper or stainless steel provided on the outer periphery of the channel tube 16.

One end portion of a ground line 33 is connected to the metal film of the intermediate layer 31 in this embodiment. As shown in FIG. 3, the other end portion of the ground line 33 is connected to a ground within the video processor 3 via a grounding circuit 37 which is composed of a capacitor 34, a resistor 35 and a coil 36. Thus, by connecting the metal film of the intermediate layer 31 of the channel tube 16 to the ground within the video processor 3, electromagnetic shield means 38 is formed for reducing the noise which occurs from the radio-frequency therapeutic device 21 that is inserted in the channel tube 16, and which mixes in the image-capturing cable 18, and also reduces crosstalk.

The following advantageous effect is obtained by the above-described structure. In this embodiment, the electrically conductive film or the mesh wires of the intermediate layer 31 of the channel tube 16 are connected to the ground within the endoscope 1. Thus, the electrically conductive film or the mesh wires of the intermediate layer 31 of the channel tube 16 are set at the ground level. By shutting off the electromagnetic field occurring around the radio-frequency therapeutic device 21 by the grounding of the intermediate layer 31 of the channel tube 16, it becomes possible to reduce crosstalk with the image-capturing cable 18 connected to the CCD 15, and to suppress disturbance of the endoscopic image displayed on the monitor 4.

In addition to the above advantageous effect, in this embodiment the resin layer 32 is formed on the outside of the intermediate layer 31 of the channel tube 16. Thus, peeling or crack of the electrically conductive film of the intermediate layer 31 can be prevented. Thereby, breakage of the electrically conductive film of the intermediate layer 31 can be prevented, and the shut-off effect of the electromagnetic field occurring around the radio-frequency therapeutic device 21 can stably be maintained.

### Third Embodiment

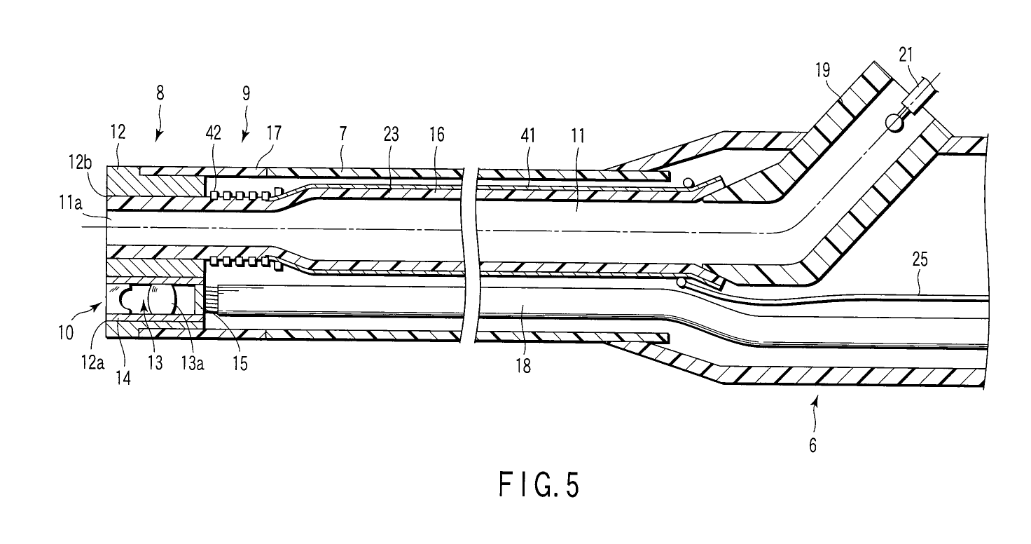

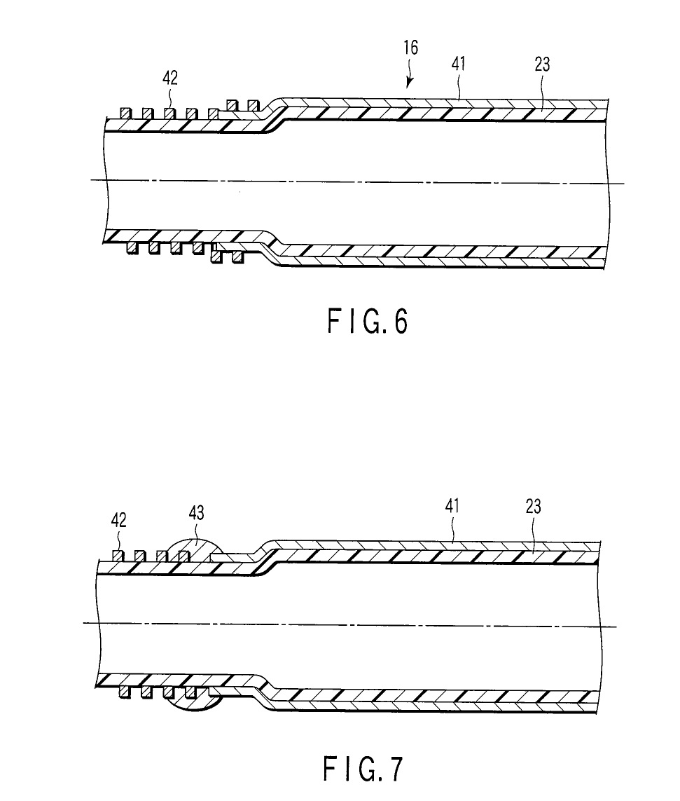

FIG. 5 and FIG. 6 show a third embodiment of the present invention. In this embodiment, the structure of the channel tube 16 of the first embodiment (see FIG. 1 to FIG. 3) is altered as described below.

In this embodiment, an outer sheath 41 is formed by providing a metal film on that part of the outer peripheral surface of the tube base layer 23, which is other than the part thereof corresponding to the bend portion 9 at the distal end portion of the channel tube 16. In addition, as shown in FIG. 6, the metal film is not formed at the part of the bend portion 9, and a coil member 42 of a metal, such as stainless steel, is wound around this part. The coil member 42 and the metal film of the outer sheath 41 are electrically connected by direct connection.

Like the first embodiment, one end portion of the lead line 25 is connected to the metal film of the outer sheath 41 of the channel tube 16. The other end portion of the lead line 25 is connected to the grounding circuit 37 provided in the video processor 3 (shown in FIG. 3). Thereby, electromagnetic shield means 27 is formed for reducing the mixing in the image-capturing cable 18 of the noise that is produced from the radio-frequency therapeutic device 21 inserted in the channel tube 16, and reducing the crosstalk.

In this embodiment, the outer sheath 41 and coil member 42 on the outer periphery of the channel tube 16 are connected to the ground within the endoscope 1. Thus, the outer sheath 41 and coil member 42 of the channel tube 16 are also grounded. Since the electromagnetic field occurring around the radio-frequency therapeutic device 21 is shut off by the grounding of the outer sheath 41 and coil member 42 of the channel tube 16, it becomes possible to reduce crosstalk with the image-capturing cable 18 connected to the CCD 15, and to suppress disturbance of the endoscopic image displayed on the monitor 4.

In addition, in this embodiment, in particular, the outer sheath 41 is formed by providing the metal film on that part of the outer periphery of the tube base layer 23, which is other than the bent portion 9 at the distal end portion of the channel tube 16, and the coil member 42 of stainless steel is wound around the part of the bent portion 9. Since there is no need to form a film on the bent portion 9, the manufacture is economical. In addition, even when the bend operation of the bend portion 9 is performed, there occurs no such situation that a metal film of the outer sheath 41 is peeled at the part of the bend portion 9, and advantageously the shield properties do not deteriorate.

FIG. 7 shows a first modification of the connection part between the coil member 42 and the metal film of the outer sheath 41 of the endoscope 1 of the third embodiment (see FIG. 5 and FIG. 6). In this modification, an electrically conductive adhesive (an adhesive containing carbon, silver powder, etc.) 43 is provided at the connection part between the coil member 42 and the metal film of the outer sheath 41. The coil member 42 and the metal film of the outer sheath 41 are connected via the electrically conductive adhesive 43.

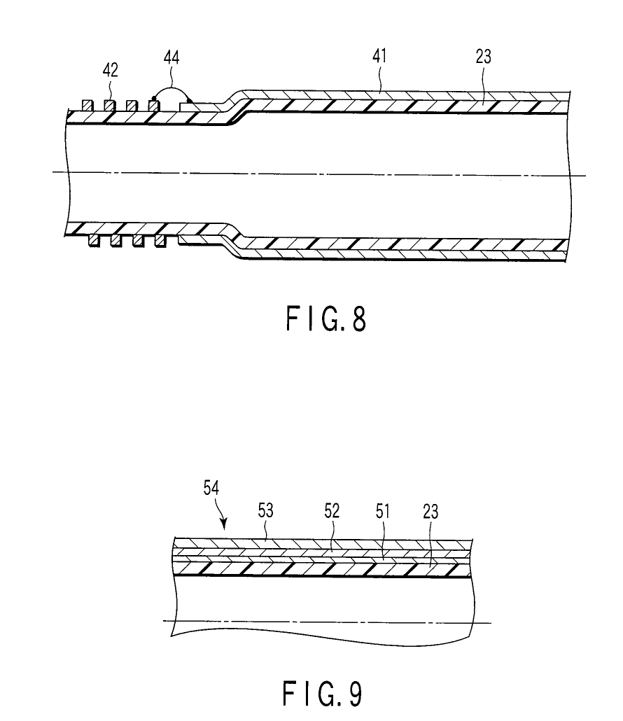

FIG. 8 shows a second modification of the connection part between the coil member 42 and the metal film of the outer sheath 41 of the endoscope 1 of the third embodiment (see FIG. 5 and FIG. 6). In this modification, a lead line 44 is provided at the connection part between the coil member 42 and the metal film of the outer sheath 41. The coil member 42 and the metal film of the outer sheath 41 are connected via the lead line 44.

In the first and second modifications, like the third embodiment, since there is no need to form a film on the bent portion 9, the manufacture is economical. In addition, even when the bend operation of the bend portion 9 is performed, there occurs no such situation that a metal film of the outer sheath 41 is peeled at the part of the bend portion 9, and advantageously the shield properties do not deteriorate.

### Fourth Embodiment

FIG. 9 shows a fourth embodiment of the present invention. In this embodiment, the structure of the channel tube 16 of the endoscope 1 of the first embodiment (see FIG. 1 to FIG. 3) is altered as described below.

In this embodiment, an underlayer 51 is formed on the outer peripheral surface of the tube base layer 23 of the channel tube 16. The underlayer 51 is formed of, e.g. titanium, chromium, or DLC (diamond-like carbon). After the outer peripheral surface of the tube base layer 23 is subjected to pre-treatment such as tetra-etching, solid sodium process, atmospheric plasma, impartment of hydrophilic properties, spreading of primer, etc., the underlayer 51 is formed by CVD, sputtering, evaporation deposition, plating, etc.

Further, a shield layer 52 is provided on the outer peripheral surface of the underlayer 51. The shield layer 52 is formed by forming a metal film of, e.g. copper, chromium, nickel or titanium by CVD, sputtering, evaporation deposition, plating, etc.

Moreover, a surface layer 53 is provided on the outer peripheral surface of the shield layer 52. The surface layer 53 is formed by forming a metal film of, e.g. gold, nickel or chromium by CVD, sputtering, evaporation deposition, plating, etc. In this example, the surface layer 53 is a layer of at least one of gold and nickel, which enhance solder wettability.

In this embodiment, the outer sheath of the channel tube 16 is formed of a stacked body 54 which comprises, in a successively stacked fashion, the underlayer 51 on the outer periphery of the tube base layer 23 of the channel tube 16, the shield layer 52 on the outer periphery of the underlayer 51, and the surface layer 53 on the outer periphery of the shield layer 52.

The following advantageous effects can be obtained by the above-described structure. In this embodiment, since the underlayer 51 is formed of chromium by sputtering with high film-formation energy, the adhesion between the outer periphery of the tube base layer 23 of the channel tube 16 and the under layer 51 can be enhanced.

Since the shield layer 52 is formed on the outer peripheral surface of the underlayer 51, electromagnetic shield properties can be imparted to the tube base layer 23, such as a PTFE tube, of the channel tube 16.

In addition, the surface layer 53 is stacked on the outer periphery of the shield layer 52, and the outermost layer of the channel tube 16 is formed as the layer of at least one of gold and nickel, which enhance solder wettability. In this case, in particular, the solder wettability of the channel tube 16 can be enhanced by the outermost surface layer 53 of the channel tube 16, which is formed of at least one of gold and nickel.

The surface layer 53 on the outer periphery of the shield layer 52 may be formed as a layer of at least one of gold, nickel and stainless steel, which enhance chemical resistance. In this case, in particular, the chemical resistance of the channel tube 16 can be enhanced by the outermost surface layer 53 of the channel tube 16, which is formed of at least one of gold, titanium and chromium. Therefore, deterioration of the channel tube 16 due to washing, disinfection and sterilization can be prevented.

### Fifth Embodiment

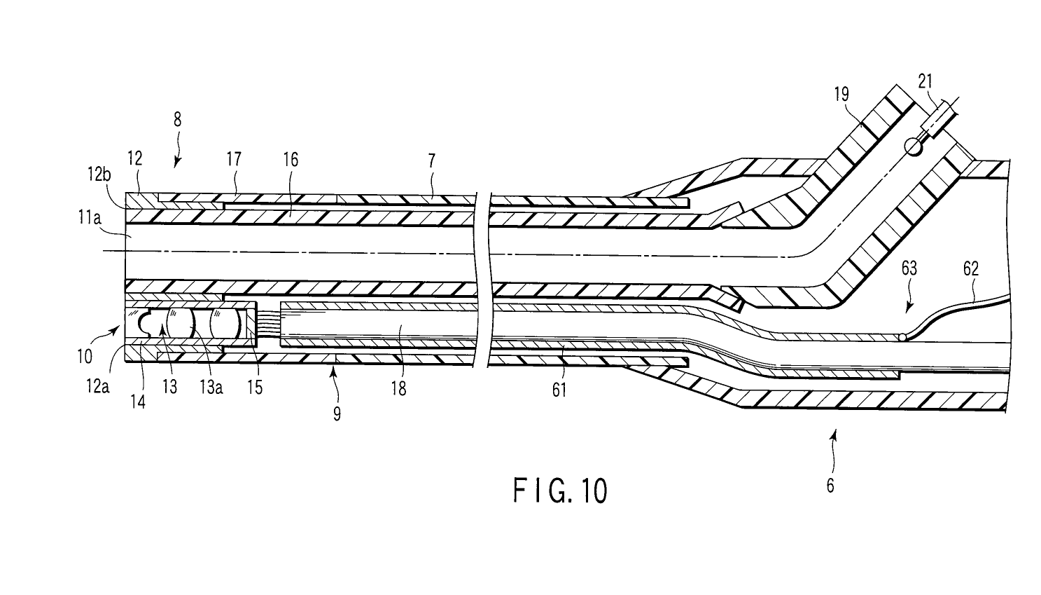

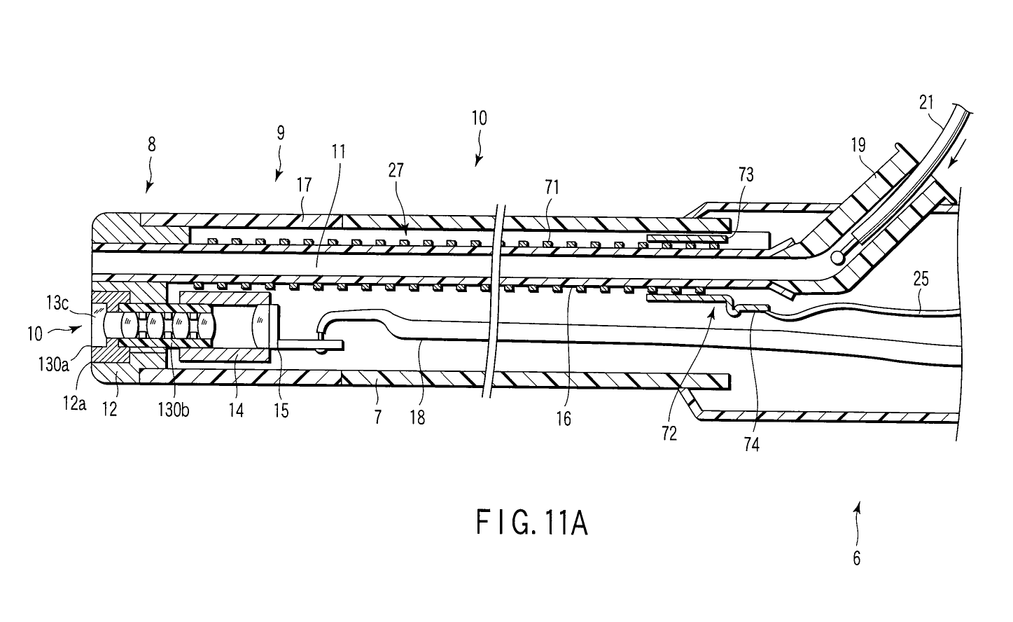

FIG. 10 shows a fifth embodiment of the present invention. In the first embodiment (see FIG. 1 to FIG. 3), the outer sheath 24 is formed by providing the metal film on the outer periphery of the tube base layer 23 of the channel tube 16. Alternatively, in the present embodiment, a metal film is formed on the outer periphery of the image-capturing cable 18 that is connected to the CCD 15, and thus an outer sheath 61 that functions as a shield is formed. In the other structural aspects, the endoscope 1 of the present embodiment is the same as that of the first embodiment. The parts common to those of the endoscope 1 of the first embodiment are denoted by like reference numerals, and a description thereof is omitted here.

One end portion of a lead line 62 is connected to the metal film of the outer sheath 61. The other end portion of the lead line 62 is connected to the grounding circuit 26 (see FIG. 3) provided in the operation section 6. Thus, by electrically connecting the lead line 62 to the ground within the endoscope 1, electromagnetic shield means 63 is formed for reducing the crosstalk of the noise in the image-capturing cable 18, which occurs from the radio-frequency therapeutic device 21 that is inserted in the channel tube 16.

The following advantageous effects can be obtained by the above-described structure. In this embodiment, since the film layer functioning as the shield layer is formed as the outer sheath 61 of the image-capturing cable 18, it becomes possible to reduce disturbance of the image of the endoscope 1 due to the noise from the radio-frequency therapeutic device 21. Further, in this embodiment, since the outer sheath 61 of the image-capturing cable 18 functions as the shield, the influence of noise from the image-capturing cable 18 can effectively be suppressed.

The metal film of the outer sheath 61 of the image-capturing cable 18 does not need to be formed over the entire length of the image-capturing cable 18. The metal film may be formed only over the range in the vicinity of at least the bend portion 9 of the insertion section 5 and the channel tube 16 in the flexible tube portion 7.

### Sixth Embodiment

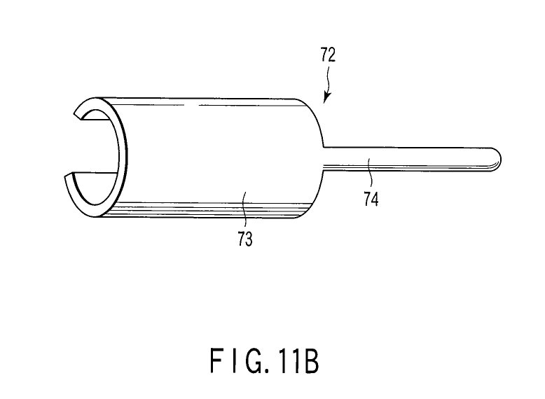

FIG. 11A and FIG. 11B show a sixth embodiment of the present invention. In this embodiment, the structure of the channel tube 16 of the third embodiment (see FIG. 5 and FIG. 6) is altered as described below.

In this embodiment, as shown in FIG. 11A, a metal film is not formed on the outer periphery of the channel tube 16, and a coil member 71 of a metal, such as stainless steel, is wound around the channel tube 16. The coil member 71 is provided not only on the bend portion 9, but also over the entirety of the channel tube 16.

A grounding connection part 72, which is formed of a metal, is attached to an operation section 6 side end portion of the coil member 71. As is shown in FIG. 11B, the grounding connection part 72 includes a coil connection portion 73 having a notch in its side part. The coil connection portion 73 is fitted on the coil member 71 and is attached so as to tighten the coil member 71. One end portion of the coil connection portion 73 is provided with a straight terminal 74. A ground line 25 is attached to the terminal 74 by soldering. Thus, the coil member 71 is electrically connected to the ground via the grounding connection part 72, ground line 25 and grounding circuit 37 of the video processor 3. Specifically, the electromagnetic shield means 27 is formed around the channel tube 16.

The coil member 71 is configured to tighten the channel tube 16. In other words, buckling of the channel 16 is prevented by the coil member 71.

The objective lens unit 13 of the observation section 10 comprises an objective lens frame 130a which holds an objective lens 13c, and a lens frame 130b which is formed of an insulating material such as resin or ceramics and holds objective lenses 13a other than the objective lens 13c. The CCD hold frame 14 that holds the CCD 15 is connected to the lens frame 130b.

Next, the operation of the above structure is described. In this embodiment, since the coil member 71 on the outer periphery of the channel tube 16 is connected to the ground, the coil member 71 shuts off the electromagnetic field that is generated around the radio-frequency therapeutic device 21. Thereby, the crosstalk in the image-capturing cable 18 connected to the CCD 15 is reduced, and the disturbance of the endoscopic image displayed on the monitor 4 is reduced.

Furthermore, in this embodiment, since there is no need to form a metal film on the outer periphery of the channel tube 16, the manufacture is economical. In addition, since the coil member 71 is wound over the entire length of the channel tube 16, buckling of the channel tube 16 will hardly occur. Hence, the thickness of the channel tube can further be reduced, and the diameter of the insertion section can be reduced.

Since a part of the objective lens unit 13, that is, the lens frame 130b which holds objective lenses 13a other than the objective lens 13c, is formed of an insulating material, the insulation from the distal-end objective lens frame 130a is enhanced. In other words, it is possible to more effectively prevent a radio-frequency leak current, which flows through the perfusate, from flowing to the CCD 15 via the CCD hold frame of the objective lens unit 13.

The present invention is not limited to the above-described embodiments. For example, a combination of the first embodiment (see FIG. 1 to FIG. 3) and the fifth embodiment (see FIG. 10) and a combination of the third embodiment (see FIG. 5 and FIG. 6) and the fifth embodiment (see FIG. 10) are effective. Needless to say, other various modifications may be made without departing from the spirit of the present invention.

### INDUSTRIAL APPLICABILITY

The present invention is effective in technical fields of manufacturing and using endoscopes, wherein an endoscopic image is captured by an imaging element such as a CCD, and a radio-frequency therapeutic device is inserted in a therapeutic device insertion channel, thereby to perform radio-frequency therapeutic treatment.

What is claimed is:

1. An endoscope comprising:
an insertion section which includes a distal end portion and a proximal end portion and is inserted in a lumen;
a therapeutic device insertion channel which is provided in the insertion section and permits passage of a therapeutic device;
an imaging section which is provided at the distal end portion of the insertion section, captures an image of a subject, converts the captured image of the subject to an electric signal, and outputs the electric signal; and
electromagnetic shield means which is provided in the therapeutic device insertion channel and suppresses, when noise is produced from the therapeutic device inserted in the therapeutic device insertion channel, the noise from mixing in the electric signal that is output from the imaging section.

2. An endoscope comprising:
an insertion section which includes a distal end portion and a proximal end portion and is inserted in a lumen;
a channel tube constituting a therapeutic device insertion channel which is provided in the insertion section and permits passage of a therapeutic device;
an imaging section which is provided at the distal end portion of the insertion section, captures an image of a subject, converts the captured image of the subject to an electric signal, and outputs the electric signal; and
a metallic part which is provided on the channel tube,
wherein the metallic part is connected to a ground on a circuit.

3. The endoscope according to claim 2, wherein the insertion section is provided with a bend portion which is bent in accordance with a user's operation, and
the metallic part includes a metallic coil member provided on a part of the channel tube, which corresponds to the bend portion, and a metal coating film provided on a part of the channel tube, which is other than the part corresponding to the bend portion, the coil member and the metal coating film being electrically connected.

4. The endoscope according to claim 2, wherein the imaging section includes an observation optical system and an imaging element, and
an image-capturing cable which is connected to the imaging element is inserted in the insertion section.

5. The endoscope according to claim 3, wherein the channel tube includes a tube base layer which is formed of a resin material, and
the metal coating film is formed of a plated coating film.

6. The endoscope according to claim 3, wherein the channel tube includes a tube base layer which is formed of a resin material, and
the metal coating film includes a film layer in which films of a plurality of kinds of metals are successively formed, and an outermost film layer which is formed of at least one of gold and nickel as an outermost sheath of the film layer.
